(19) United States
(12) Patent Application Publication
(10) Pub. No.: US 2007/0088007 A1
(43) Pub. Date: Apr. 19, 2007

(54) Method for preparing analogue of vitamin D

(75) Inventors: Chze Siong Ng, Taoyuan (TW); Ching-Peng Wei, Taoyuan (TW)

Correspondence Address: LOWE HAUPTMAN BERNER, LLP, 1700 DIAGONAL ROAD, SUITE 300, ALEXANDRIA, VA, 22314, US

(73) Assignee: Formosa Laboratories, Inc., Taoyuan (TW)

(21) Appl. No.: 11/477,698

(22) Filed: Jun. 30, 2006

(30) Foreign Application Priority Data
Oct. 14, 2005 (TW) 094135931

(51) Int. Cl. A61K 31/59; C07C 401/00

(52) U.S. Cl. 514/167; 552/653

(57) ABSTRACT

A method for preparing analogues of C1,C24-dihydroxy-vitamin D is disclosed. Especially the method for preparing calcipotriol and tacalcitol from a starting material of Vitamin D2 is disclosed here. Calcipotriol (compound 1(a)) and tacalcitol (compound 1(b)) can be synthesized by the method of the present invention. Moreover, only nine steps are needed for the synthesis of calcipotriol using the method. Likewise, only ten steps are needed for the synthesis of tacalcitol by the present method. Hence, the present method, with less process steps and higher yields, represents an improvement over the conventional methods.

### BACKGROUND OF THE INVENTION

1. Field of the Invention

The present invention relates to a method for preparing an analog of C1-hydroxyl-C24-hydroxyl-vitamin D, and, more particularly, to a method for preparing calcipotriol and tacalcitol with a starting material—vitamin D2.

2. Description of Related Art

It is known that calciportriol and tacalcitol are analogues of vitamin D. Moreover, calcipotriol (e.g. (5Z,7E,22E,24S)-24-cyclopropyl -9,10-secochola-5,7,10(19),22-tetraene-1α,3β,24-triol) shows strong activity in inhibiting undesirable proliferation of epidermal keratinocytes. On the other hand, since tacalcitol can increase the absorption of calcium cation in the intestine, tacalcitol can be used for treating osteoporosis, and bone disorders resulted from renal insufficiency, hypoparathyoidism, or rickets.

Many conventional methods for manufacturing calciportriol or tacalcitol have been disclosed. For example, calciportriol can be X synthesized from the starting material vitamin D2. After the starting vitamin D2 is protected by double cyclization, deprotected through ring-opening, and oxidized with stereo selectivity, calcipotriol can be synthesized. However, this conventional manufacturing method needs special oxidants for reaction. Besides, the required 12 steps of this conventional manufacturing method are very complicated. Hence, the conventional method illustrated above is not suitable for mass-production. (WO8700834; Tetrahedron ,43,4609(1987)

Another method for manufacturing calciportriol calls for modifying the substitution at C22 of vitamin D2 into a sulfone functional group. The sulfone derivative is then coupled with a side-chain aldehyde of a stereoisomerically pure structure. A total of 15 steps is required for the preparation of calcipotriol. Furthermore, the synthesis of the stereoisomerically pure side-chain aldehyde is difficult, and the final yield of the reaction is therefore affected. Therefore, this method is not convenient for mass production either. (WO 03/087048A2, 2003)

Tacalcitol can also be prepared from Fucosterol through selective ring-opening, thermal rearrangement, repeated oxidation and reduction. The yield of the reported method is low, and at least 12 steps are required for the synthesis. (U.S. Pat. No. 4,022,891. 1977) In yet another reported method, tacalcitol is synthesized by coupling two independent moieties (JP 7,112,968, 1995). The 24-step method of the disclosure JP 7,112,968 is complicated, and again suffers from low yields.

Since the conventional methods for preparing analogues of calcipotriol and tacalcitol are complicated, and suffer from low-yields, it would be desirable to develop shorter and more efficient synthetic processes, for preparing C1-hydroxyl-C24-hydroxyl-vitamin D.

### SUMMARY OF THE INVENTION

The present invention provides a simplified method for preparing analogues of C1-hydroxyl-C24-hydroxyl-vitamin D, especially for preparing calciportriol and tacalcitol with vitamin D2 as starting material. One advantage of the present invention is the reduced number of steps. For example, only nine steps are required for preparing calcitpotriol (compound 1(a)) using the method of the present invention. Similarly, only ten steps are required for preparing tacalcitol (compound 1(b)). Therefore, the method of the present invention for the preparation of Vitamin D analogues represents an improvement over the conventional methods of lengthy pathway and low yields.

The method for preparing an analogues of C1-hydroxyl-C24-hydroxyl-vitamin D of the present invention comprises the following steps: (a) oxidizing a starting material of formula (1): 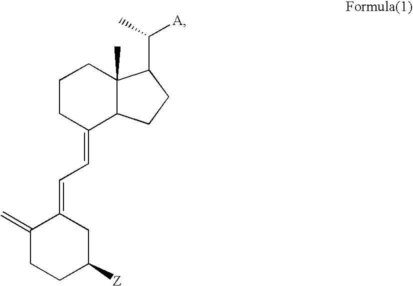
by an oxidant to form a mixture of isomers, wherein A is 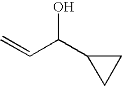
or 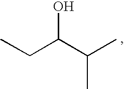
and Z is a protected hydroxyl group; and (b) photo-isomerization and deprotecting the mixture of isomers to form an analogue of C1(α)-hydroxyl-C24-hydroxyl-vitamin D or C1(β)-hydroxyl-C24-hydroxyl-vita D of formula (2): 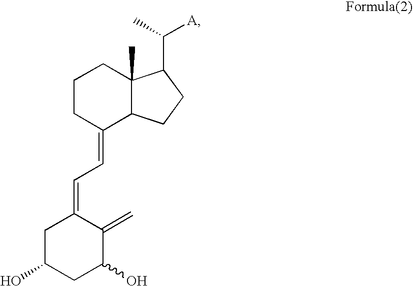
wherein the oxidant is a selenium dioxide or a selenite ester of formula (3):
R⁶O—Se(O)—OR⁷ (3).

In formula (3), R⁶ and R⁷ are individually a hydrogen, C₁-C₉ alkyl, C₁-C₉ aralkyl, or the combination thereof, and R⁶ and R⁷ are identical or different. The selenite ester used in the present invention can be prepared through the method described in U.S. Pat. No. 4,263,215. Preferably, R⁶ and R⁷ in formula (3) of the selenite ester used in the present invention are individually hydrogen, C₁-C₄ alkyl. The Z in formula (1) of the present invention is a protected hydroxyl group. Preferably, Z is an ether or ester. More preferably, Z is t-butyldimethylsilyloxy.

The oxidation of the method of the present invention can be achieved by an oxidant and optionally a co-oxidant. Preferably, the starting material is oxidized in the presence of a co-oxidant in step (a). The co-oxidant is not limited. Preferably, the co-oxidant of the present invention is a metal salt of a peracid, an alkyl hydroperoxide in which the alkyl moiety contains from 4 to 16 carbon atoms, a non-aromatic tertiary amine oxide, or a combination thereof. More preferably, the coxidant is sodium metaperiodate, or N-methylmorpholin N-oxide.

In one of the aspect of the method of the present invention, the starting material is oxidized in the presence of the oxidants and co-oxidants. In one of the preferred embodiment of the present invention, the oxidant is selenium dioxide, and the co-oxidant is N-methylmorpholine N-oxide. In another preferred embodiment of the present invention, the starting material of formula (1) is oxidized in the presence of an oxidant, a co-oxidant, and a base dissolved in an organic solvent. The base of the present invention is not limited. Preferably, the base is alkylamine, heterocyclic amine, or the combination thereof. In one of the preferred example of the present invention, the base is triethylamine (TEA). In another preferred example of the present invention, the base is imidazole or a derivative thereof

Furthermore, the organic solvent of the oxidation of the method of the present invention is not limited. Preferably, the organic solvent of the present invention is alkanol having 1 to 9 carbon atoms, haloalkane having 1 to 9 carbon atoms, arylalkane having 6 to 9 carbon atoms, or the combination thereof. In the preferred examples of the present invention, the organic solvent is methanol, dichloromethane, acetonitrile, or a combination thereof.

In one aspect of the present invention, the starting material of formula (1) is not limited with respect to the stereochemistry at C24 position. The starting material of formula (1) can be a single optical isomer, or a mixture of epimers at C24 position. The reaction site of oxidation on the starting material of formula (1) is on the C1 site. Therefore, after the oxidation, the same situation applied to the subsequent photo-isomerization and deprotection reaction. The stereochemistry at C24 position is not limited while performing the photo-isomerization reaction at C₅-C₆ double bond, and the deprotection reaction at the C3 position.

In one aspect of the present invention, the starting material of formula (1) is [5E,7E,22E,24S]-24-cyclopropyl-3β-(tert-butyldimethylsilyloxy)-9,10-secochola-5,7,10(19),22-tetraene-24-ol (de>95%). The mixture of the isomers after oxidization is [5E,7E,22E,24S]-24-cyclopropyl-3β-(tert-butyldimethyl-silyloxy)-9,10-secochola-5,7,10(19),22-tetraene-1(α,β),24-diol, which are subjected to photo-isomerization and deprotection to give (5Z,7E,22E,24S)-24-cyclopropyl-9,10-secochola-5,7,10(19),22-tetraene-1(α,β),3β,24-triol.

In another aspect of the present invention, the starting material of formula (1) is [5E,7E,24R]-24--isopropyl-3β-(tert-butyldimethylsilyloxy)-9,10-secochola-5,7,10(19)-triene-24-ol. The mixture of the isomers after oxidization is [5E,7E,24R]-24-isopropyl-3β-(tert-butyl dimethylsilyloxy)-9,10-secochola-5,7,10(19)-triene-1(α,β),24-diol, which are subjected to photo-isomerization and deprotection to give (5Z,7E,24R)-24-isopropyl-9,10-secochola-5,7,10(19)-triene-1(α,β),3β24-triol.

In one aspect of the present invention, the sequence of the photo-isomerization and deprotection after the oxidation is not limited. In other words, the photo-isomerization can be achieved before the deprotection or vice versa.

In one aspect of the present invention, the starting material of formula (1) is [5E,7E,22E,24S]-24-cyclopropyl-3β-(tert-butyldimethylsilyloxy)-9,10-secochola-5,7,10(19),22-tetraene-24-ol, which is oxidized to give [5E,7E,22E,24S]-24-cyclopropyl-3β-(tert-butyldimethylsilyloxy)-9,10-secochola-5,7,10(19),22-tetraene-1(α,β),24-diol. After further deprotection and photo isomerization are achieved, the product of the analogue of vitamin D of the present invention is (5Z,7E,22E,24S)-24-cyclopropyl-9,10-secochola-5,7,10(19), 22-tetraene-1(α,β),3β,24-triol.

In another aspect of the present invention, the starting material of formula (1) is [5E,7E,24R]-24-isopropyl-3β-(tert-butyldimethylsilyloxy)-9,10-secochola-5,7,10(19)-triene-24-ol, which is oxidized to give [5E,7E,24R]-24-isopropyl-3β-(tert-butyldimethylsilyloxy)-9,10-secochola-5,7,10(19)-triene-1(α,β),24-diol. An isomerization is performed on the double bound between C5 and C6 of the product by photo isomerization. Than a deprotection process is subsequently performed. Then the protection group tert-butyl dimethylsilyloxyl of the hydroxyl group on C3 is released by deprotection to from C3 hydroxyl group. After further deprotection and photo isomerization are achieved, the product of the analogue of vitamin D of the present invention is (5Z,7E,24R)-24-isopropyl-9,10-secochola-5,7,10(19)-triene-1(α,β), 3β,24-triol.

The photo-isomerization can be achieved by any photo isomerization method. Preferably, the photo-isomerization is achieved by illuminating with a photosensitizer. The photosensitizer used in the method of the present invention is not limited. Preferably, the photosensitizer can be anthracene and its derivatives, phenazine and its derivatives, acridine and its derivatives, or the combination thereof More preferably, the photosensitizer is anthracene and the derivatives thereof.

The major center of the deprotection of the method of the present invention is the protective group of the hydroxyl group on C3 of the analogue of vitamin D. Hence, the deprotection of the method of the present invention can be any deprotection step for the protective group of a hydroxyl group. In one aspect of the present invention, the protective group of the hydroxyl group on C3 of the analogue of vitamin D is tert-butyldimethylsililoxy. Hence, the deprotection reagent for the deprotection of the protective group of the hydroxyl group on C3 of the analogue of vitamin D can be a quaternary amine. Preferably, the quaternary amine is tetra-n-butylammonium fluoride.

In addition, the present invention also provides a method for preparing an analogue of C1α-hydroxyl-C3-hydroxyl-C24-hydroxyl-vitamin D and an analogue of C1β-hydroxyl-C3-hydroxyl-C24-hydroxyl-vitamin D. The method of the present invention comprises the following steps: (a) oxidizing a starting material of formula (1): 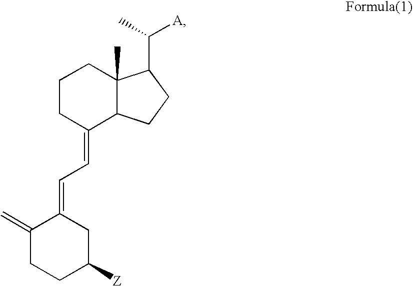
by an oxidant to form a mixture of isomers, wherein A is 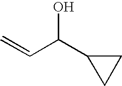
or 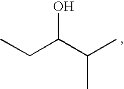
and Z is a protection group of a hydroxyl group; (b) photo-isomerizing and deprotecting the mixture of isomers, and forming a mixture of complexes with a ligand, wherein a mixture of the complexes is formed after deprotection, and the ligand is alkyl boronic acid or aryl boronic acid; and (c) separating analogue of the C1α-hydroxyl-C3-hydroxyl-C24-hydroxyl-vitamin D, and the analog of C1β-hydroxyl-C3-hydroxyl-C24-hydroxyl-vitamin D complexed with the ligand from the mixture of the complexes. The oxidant is selenium dioxide or a selenite ester of formula (3)
R⁶O—Se(O)—OR⁷ (3)
wherein R⁶ and R⁷ are individually hydrogen, C₁-C₉ alkyl, C₁-C₉ aralkyl, or the combination thereof, and R⁶ and R⁷ are identical or different.

In the method of the present invention, the complex is formed after the deprotection is achieved. The aryl boronic acid can be any aryl boronic acid. Preferably, the aryl boronic acid is phenylboronic acid. Moreover, the major sites for complexing to the boronic acid are the diols on the C1, and C3 site of the analogue of C1β-hydroxyl-C3-hydroxyl-C24-hydroxyl-vitamin D. Actually, the diol on the C1, and C3 site of the analogue of C1β-hydroxyl-C3-hydroxyl-C24-hydroxyl-vitamin D form a cyclic boronate ester with the boronic acid. Hence, the complexation can be proceeded with the cis-isomer, the trans-isomer of the C24 hydroxyl site of the C1β-hydroxyl-C3-hydroxyl-C24-hydroxyl-vitamin D, or the mixture thereof.

In one aspect of the method for preparing an analogue of C1α-hydroxyl-C3-hydroxyl-C24-hydroxyl-vitamin D and an analog of C1β-hydroxyl-C3-hydroxyl-C24-hydroxyl-vitamin D of the present invention, the analog of the C1β-hydroxyl-C3-hydroxyl-C24-hydroxyl-vitamin D to be complexed with the boronic acid is (5Z,7E,22E,24R)-24-cyclopropyl-9,10-secochola-5,7,10(19),22-tetraene-1β,3β,24-triol, (5Z,7E,22E,24-S)-24-cyclopropyl-9,10-secochola-5,7,10(19),22-tetraene-11P,3 P,24-triol, or the combination thereof.

In another aspect of the method for preparing an analogue of C1α-hydroxyl-C3-hydroxyl-C24-hydroxyl-vitamin D and an analogue of C1β-hydroxyl-C3-hydroxyl-C24-hydroxyl-vitamin D of the present invention, the analogue of the C1β-hydroxyl-C3-hydroxyl-C24-hydroxyl-vitamin D to be complexed with the boronic acid is (5Z,7E,24R)-24-isopropyl-9,10-secochola-5,7,10(19)-triene-1β,3β,24-triol, (5Z,7E,24S)-24-isopropyl-9,10-secochola -5,7,10(19)-triene-1β,3β,24-triol, or the combination thereof. In another aspect of the method for preparing an analogue of C1α-hydroxyl-C3-hydroxylC24-hydroxyl-vitamin D and an analogue of C1β-hydroxyl-C3-hydroxyl-C24-hydroxyl-vitamin D of the present invention, the analogue of the C1β-hydroxyl-C3-hydroxyl-C24-hydroxyl-vitamin D to be complexed with the boronic acid ligand is (5E,7E,22E,24R)-24-cyclopropyl-9,10-secochola-5,7,10(19),22-tetraene-1β,3β,24-triol, (5E,7E, 22E,24S)-24-cyclopropyl-9,10-secochola-5,7,10(19),22-tetraene-1β,3β,24-triol, or the combination thereof

The sequence of the photo-isomerization, the deprotection, and the formation of a complex is not limited. Basically, the formation of complexes is proceeded after deprotection is finished. In a preferred aspect of the method for preparing an analogue of C1α-hydroxyl-C3-hydroxyl-C24-hydroxyl-vitamin D and an analogue of C1β-hydroxyl-C3-hydroxyl-C24-hydroxyl-vitamin D of the present invention, the photo-isomerization is proceeded after the deprotection and the formation of a complex are achieved.

In addition, the present invention also provides a method for separating an analogue of C1(α,β)-hydroxyl-C24-hydroxyl-vitamin D. The method comprises the following steps: providing a mixture of isomers of the analogue of 1,3,24-trihydroxyl-vitamin D; reacting the mixture of isomers of the analogue of 1,3,24-trihydroxyl-vitamin D with a ligand to form a ring-structured complex, wherein the lignad is alkyl boronic acid or aryl boronic acid, and separating the ring-structured complexes of the analogue of 1,3-cis-C24-trihydroxyl-vitamin D and the analogue of 1,3-trans-24-trihydroxyl-vitamin D to obtain the analogue of the C1β, C3,C24-trihydroxyl-vitamin D and the analogue of the C1α,C3,C24-trihydroxyl-vitamin D, individually. The analogue of 1,3,24-trihydroxyl-vitamin D to form a ring-structured complex is an analogue of 1,3-cis-C24-trihydroxyl-vitamin D.

The isomer of the analogue of 1,3,24-trihydroxyl-vitamin D is a compound having the structure of (2) 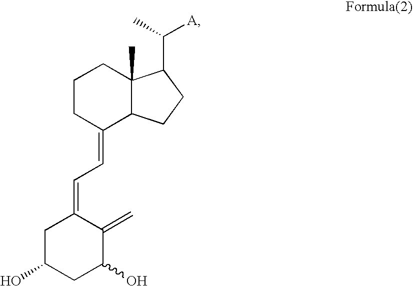
or having the structure of (4) 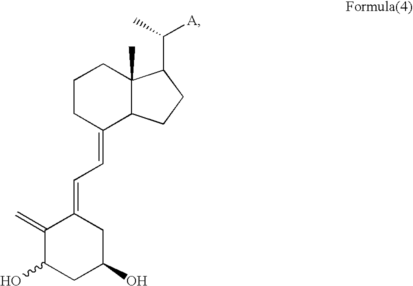
wherein A is 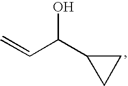
or

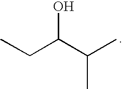

Other objects, advantages, and novel features of the invention will become more apparent from the following detailed description when taken in conjunction with the accompanying drawings.

### DETAILED DESCRIPTION OF THE PREFERRED EMBODIMENT

The synthetic route of the method of the compound starts from the starting material vitamin D2. A common intermediate (compound 5) is synthesized first according to the synthetic route of the method of the present invention. Then, two series of vitamin D analogues having various substituted group on C22 carbon are synthesized.

The synthetic route 1 shown in the following scheme is the synthetic route of the preferred embodiment of the present invention. However, the method for synthesizing analogues of vitamin D2 of the present invention is not limited by Synthetic route 1 as illustrated. Actually, the intermediate compound 5 used here can be prepared through the steps described in synthetic route 1, or by other conventional methods.

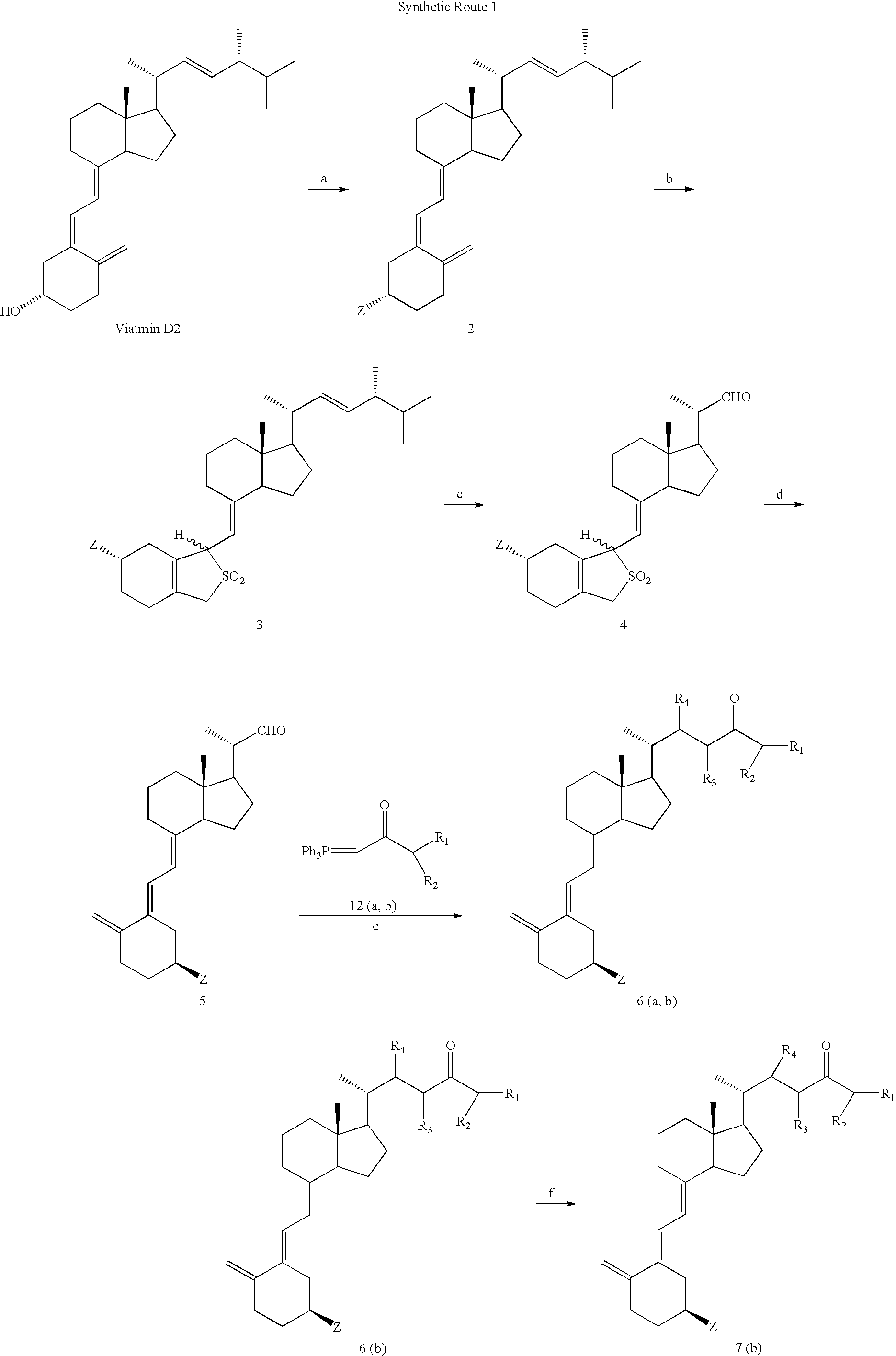

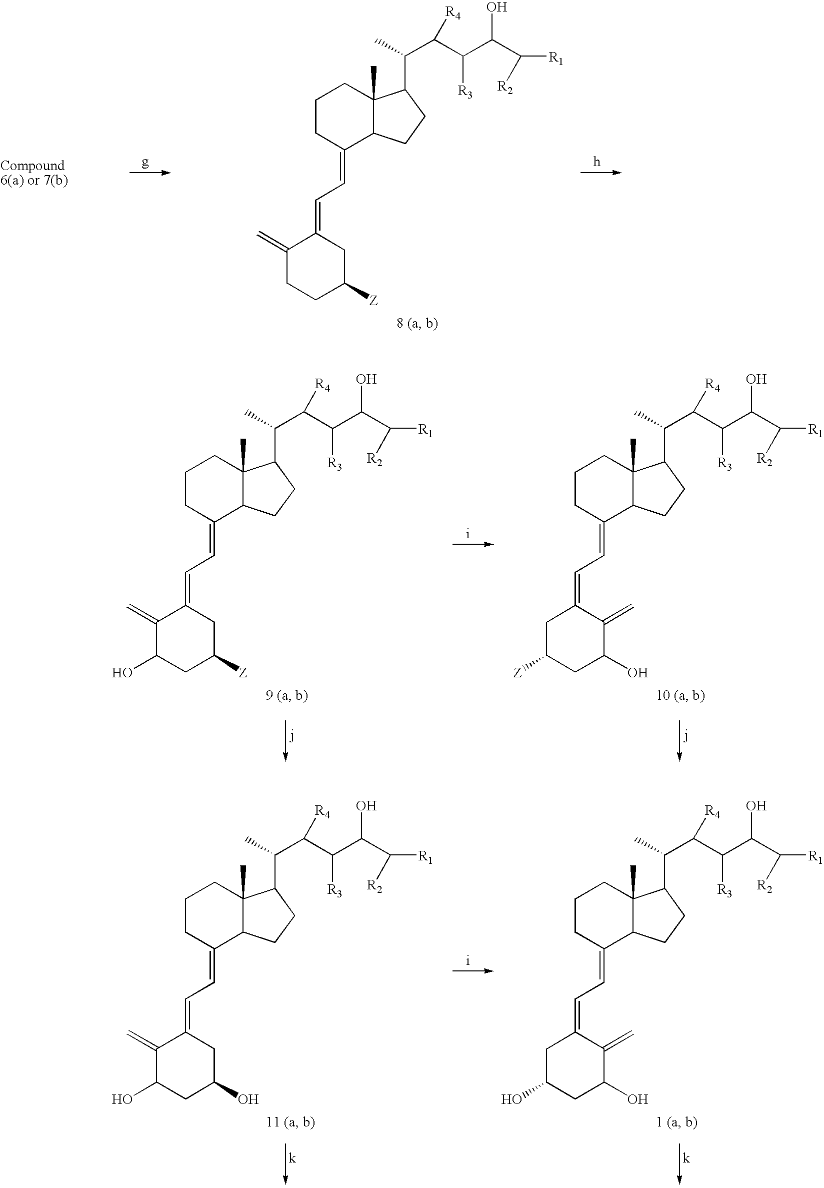

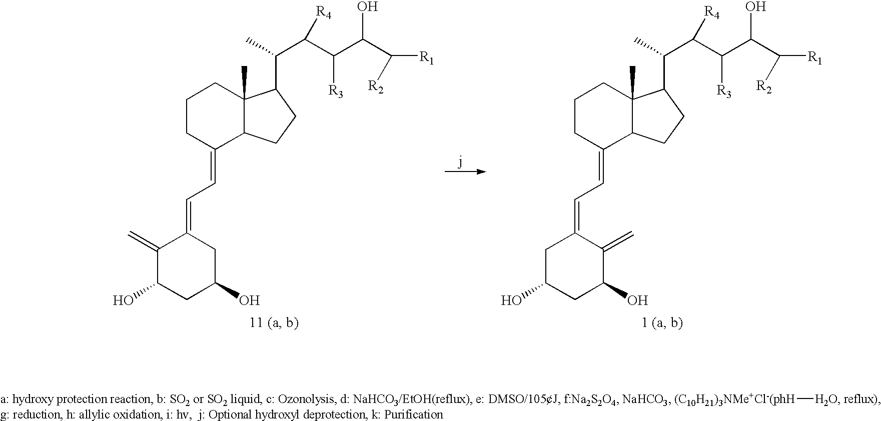

The designated structural units of compounds 1 to 12, i.e. the analogues of vitamin D, prepared in the preferred examples of the present invention according to Synthetic route 1 are listed in table 1.

TABLE 1

| compound | R₁ | R₂ | R₃ | R₄ |
|---|---|---|---|---|
| 2 | — | — | — | — |
| 3 | — | — | — | — |
| 4 | — | — | — | — |
| 5 | — | — | — | — |

| | | |
|---|---|---|
| 6(a) | —(CH₂)₂— | bond |

| | | | |
|---|---|---|---|
| 6(b) | CH₃ | CH₃ | bond |

| | | | | |
|---|---|---|---|---|
| 7(b) | CH₃ | CH₃ | H | H |

| | | |
|---|---|---|
| 8(a) | —(CH₂)₂— | bond |

| | | | | |
|---|---|---|---|---|
| 8(b) | CH₃ | CH₃ | H | H |

| | | |
|---|---|---|
| 9(a) | —(CH₂)₂— | bond |

| | | | | |
|---|---|---|---|---|
| 9(b) | CH₃ | CH₃ | H | H |

| | | |
|---|---|---|
| 10(a) | —(CH₂)₂— | bond |

| | | | | |
|---|---|---|---|---|
| 10(b) | CH₃ | CH₃ | H | H |

| | | |
|---|---|---|
| 11(a) | —(CH₂)₂— | bond |

| | | | | |
|---|---|---|---|---|
| 11(b) | CH₃ | CH₃ | H | H |

| | | | |
|---|---|---|---|
| 12(a) | —(CH₂)₂— | — | — |

| | | | | |
|---|---|---|---|---|
| 12(b) | CH₃ | CH₃ | — | — |

| | | |
|---|---|---|
| 1(a) | —(CH₂)₂— | bond |

| | | | | |
|---|---|---|---|---|
| 1(b) | CH₃ | CH₃ | H | H |

### EXAMPLE 1

### Preparation of Compound 2 (Z=t-BuMe₂SiO)

Vitamin D2 (2 kg, 5.04 mol), tert-butyldimethylsilyl chloride (1.16 kg, 7.70 mol), and imidazole (1.03 kg, 15.15 mol) are dissolved in dichloromethane (20 L). The mixture is stirred at room temperature for 2 hours. After the reaction is complete, the reaction mixture is checked by TLC (with an eluent of 10% ethyl acetate in hexane). The reaction mixture is washed with water (6 L), sodium chloride aqueous solution (6 L), and water (6 L) in sequence. The organic layer is concentrated under reduced pressure, and compound 2 (2.5 kg) is obtained. The product obtained can be used in the subsequent reaction without further purification.

Compound 2 (Z=t-BuMe₂SiO): ¹H NMR (200 MHz, CDCl₃) δ 0.07 (s, 6H), 0.56 (s, 3H), 3.77-3.86 (m, 1H), 4.14 (s, 1H), 4.78 (s, 1H), 5.09-5.28 (m, 2H), 6.02 (d, 1H, J=11.2 Hz), 6.17 (d, 1H, J=11.2 Hz).

### EXAMPLE 2

### Preparation of Compound 3 (Z=t-BuMe₂SiO)

Compound 2 (2.50 kg, 4.89 mol) prepared in example 1 is dissolved in dichloromethane (20 L) to form a solution. The solution is then added to a saturated sulfur dioxide (SO₂) aqueous solution (10 L), and then stirred at room temperature for 2 hours. After the reaction is complete, the reaction solution is checked by TLC (with an eluent of 10% ethyl acetate in hexane). The reaction solution is heated to remove SO_{2.} The residue after evaporation is dissolved in ethyl acetate (6.3 Kg). The resulted ethyl acetate solution is washed with water, and concentrated to give compound 3 (2.72 kg). The product obtained can be used for the subsequent reaction without further purification.

### EXAMPLE 3

### Preparation of Compound 4 (Z=t-BuMe₂SiO)

Compound 3 (2.72 kg, 4.73 mol) prepared in example 2 is dissolved in a mixture of dichloromethane (25 L) and methanol (2.5 L) to form a solution. The solution is cooled to −60° C., and ozone is introduced to it. The reaction solution is monitored and checked by TLC (with an eluent of 30% ethyl acetate in hexane). When the starting material is depleted, the reaction is quenched.

Then nitrogen is introduced to the solution, and dimethyl sulfide (1.6 kg, 25.81 mol) is added to the solution subsequently. The resulted solution is heated to room temperature slowly to quench the extra ozone. Dichloromethane (13.2 L) is added to the quenched solution. The resulted mixture is washed with water, and concentrated to give compound 4 (2.20 kg).

### EXAMPLE 4

### Preparation of Compound 5 (Z=t-BuMe₂SiO)

Compound 4 (2.20 kg, 4.34 mol) prepared in example 3 is dissolved in 95% ethanol (16 L). Sodium hydrogen carbonate(2.0 Kg, 23.81 mol) is added to the ethanol solution and the mixture is stirred under an atmosphere of nitrogen, and refluxed for 120 minutes. After the reaction is complete based on TLC analysis (with an eluent of 10% ethyl acetate in hexane), the solution is washed with water (L), and concentrated to give compound 5 (1.73 kg ). The product obtained can be used for the subsequent reaction without further purification.

Compound 5 (Z=t-BuMe₂SiO): ¹H NMR (200 MHz, CDCl₃) δ3.81-3.83 (m, 1H), 4.62 (s, 1H), 4.90 (s, 1H), 5.85 (d, 1H, J=11.2 Hz), 6.46 (d, J=11.2 Hz), 9.52-9.58 (m, 1H).

### EXAMPLE 5

### Preparation of Compound 12(b)

Methyl isopropyl ketone (21.5 g, 249.6 mol) is dissolved in methanol (250 mL). The methanol solution is stirred and cooled to a temperature below 10° C. Bromine (39.9 g, 250 mmol) is added to the methanol solution and reacted at a temperature below 30° C. for 60 minutes. After the reaction is complete, aqueous sodium carbonate solution (250 mL) is added to quench the reaction. The quenched solution is stirred for 15 minutes. Then the resulted solution is extracted three times with hexane (170 mL×3). The organic layer is collected, washed with aqueous sodium carbonate solution, dried with MgSO₄, and concentrated to obtained bromomethyl isopropyl ketone (25.5 g).

Bromomethyl isopropyl ketone: ¹H NMR (200 MHz, CDCl₃) δ1.08 (d, J=7.0 Hz, 6 H), 2.96 (hepta, J=7.0 Hz, 1H), 3.96 (s, 2H).

The intermediate bromomethyl isopropyl ketone (25.5 g) obtained above is dissolved in toluene (132 mL) and stirred under nitrogen. To the above solution is added slowly a second solution of triphenylphosphine (43 g, 164 mmol) in toluene (88 mL). The combined solution is stirred at room temperature for 17 hours for the reaction to go to completion, and then filtered to obtain solids. The obtained solids are dissolved in dichloromethane (182 mL), followed by the addition of 2N sodium hydroxide aqueous solution (60 mL). The mixture is kept stirring at room temperature for 1 hour. The reaction mixture is then washed with water, and concentrated to give compound 12(b) (37.8 g).

Compound 12(b): ¹H NMR (200 MHz, CDCl₃) δ1.15 (d, J=6.9 Hz, 6H) 2.45(hepta, J=6.9 Hz, 1 H) 3.66(bd, J=26.5Hz, 1 H) 7.31-7.69 (m, 15H).

### EXAMPLE 6

### Preparation of Compound 6(a, b)

(6-1-1) Preparation of Compound 6(a) (Z=t-BuMe₂SiO)

Compound 5 (5 g, 11.29 mmol) prepared in example 4, and compound 12(a) (7.8 g, 22.65 mmol) (prepared by the method described in WO8700834) are added to dimethyl sulphoxide (DMSO) (20 mL) to form a solution, which is heated to 95° C. for 90 minutes for the coupling reaction to proceed and then to 120° C. for 120 minutes for the reaction to go to completion. The reaction mixture is then cooled, followed by addition of water and then extraction with ethyl acetate. The combined ethyl acetate extracts are washed with water, and concentrated to give a crude product, which is purified through a chromatographic column (with an eluent of 10% ethyl acetate in hexane) to give compound 6(a) (4.0 g).

Compound 6(a): λₘₐₓ=266 nm. ¹H NMR (200 MHz, CDCl₃) δ 0.54(s, 3H), 3.80(m, 1H), 4.56(bs, 1H), 4.84(bs, 1H), 5.78(d, J=11.4 Hz, 1H), 6.09(d, J=15.6 Hz, 1H), 6.39(d, J=11.4 Hz, 1H), 6.70(dd, J-15.6 Hz & 8.8 Hz, 1H).

(6-1-2) Preparation of Compound 6(a) (Z=t-BuMe₂SiO)

The steps for the preparation are similar to those described in example (6-1-1) except that the solvent DMSO is replaced by ethanol and the reaction condition is changed to refluxing to complete the reaction. In the present example, compound 5 (1.73 kg, 3.91 mol) prepared in example 4 and compound 12(a) (2.5 kg, 7.48 mmol) are used for the reaction. After purification by column chromatography, compound 6 (a) (1.90 kg) is obtained.

(6-2) Preparation of Compound 6(b) (Z=t-BuMe₂SiO)

The steps for the preparation are similar to those described in example (6-1-1) except that the reactant for Wittig reaction, i.e. compound 12(a), is replaced by compound 12(b) prepared in example 5. In the present example, compound 5 (5 g, 11.29 mmol) prepared in example 4, and compound 12(b) (7.8 g, 22.48 mmol) prepared in example 5 are used for reaction. After purification by column chromatography, compound 6 (b) (3.8 g) is obtained.

### EXAMPLE 7

### Preparation of Compound 7(b) (Z=t-BuMe₂SiO)

Compound 6(b)(2.5 g, 4.89 mmol), sodium hydrogen carbonate (6.25 g, 74.39 mmol), sodium dithionite (Na₂S₂O₄) (6.25 g, 35.90 mmol), and methyltridecylammonium chloride (3.13 g) are dissolved in a mixed solution of toluene (125 mL) and water (125 mL). Then the mixed solution is heated to a temperature ranging from 80-85° C. under nitrogen atmosphere. The mixed solution is remained in the same temperature and nitrogen atmosphere illustrated above with stirring for 4 hours to react. After the reaction is completed, the reacted mixture is cooled and separated into layers. The organic layer is washed with water, and concentrated and concentrated to give a crude product, which is purified through a chromatographic column (with an eluent of 10% ethyl acetate in hexane) to give compound 7 (b) (0.5 g).

### EXAMPLE 8

### Preparation of Compound 8(a, b)

(8-1) Preparation of Compound 8(a) (24S, Z=t-BuMe₂SiO) and Compound 8(a) (24R, Z=t-BuMe₂SiO)

Methanol (4.0 L) is added to the THF solution (16.0 L) of compound 6(a)(Z=t-BuMe₂SiO)(1.90 kg, 3.73 mol). The mixed solution is kept in a low temperature with stirring. Sodium borohydride (150 g, 3.97 mol) is added to the mixed solution slowly. After the reaction is completed (the reacted solution is monitored and checked by TLC (with an eluent of 10% ethyl acetate in hexane)), the reacted solution is concentrated. Then the concentrated residue is added with ethyl acetate and water for extraction. The organic layer is washed with water, and concentrated and concentrated to give a crude product (compound 8(a), (2.62 kg)), which is purified through a chromatographic column (with an eluent of 6% ethyl acetate in hexane) to give compound 8(a) (24R, Z=t-BuMe₂SiO, d.e.=88%) (720 g) and compound 8(a) (24S, Z=t-BuMe₂SiO, d.e.=99%) (446.2 g).

Compound 8(a)(24R, Z=t-BuMe₂SiO): λₘₐₓ=266 nm. ¹H NMR (200 MHz, CDCl₃) δ 3.42(br, 1H) 3.80(br, 1H), 4.59(s, 1H), 4.87 (s, 1H), 5.3-5.48(m, 2H), 5.80(d,1H,J=11.52 Hz), 6.41(d, 1H, J=11.26 Hz).

Compound 8(a) (24S, Z=t-BuMe₂SiO): λₘₐₓ=266 nm. ¹H NMR (200 MHz, CDCl₃) δ 3.40(br,1H), 3.80(br, 1H), 4.61(s, 1H), 4.89 (s, 1H), 5.41-5.45(m,2H), 5.82(d,1H,J=11.5 Hz), 6.44(d,1H,J=11.48 Hz.

(8-2) Preparation of Compound 8(b) (24S, Z=t-BuMe₂SiO) and Compound 8(b) (24R, Z=t-BuMe₂SiO)

The steps for the preparation are similar to those described in example (8-1) except that the starting material, i.e. compound 6(a), for the reaction is replaced bycompound 7(b) prepared in example 7. In the present example, compound 7(b) (0.5 g, 0.97 mmol) prepared in example 7 is used for reaction. After purification by column chromatography, compound 8(b) (24S, Z=t-BuMe₂SiO) (170 mg) and compound 8(b) (24R, Z=t-BuMe₂SiO) (95 mg) are obtained.

### EXAMPLE 9

### Preparation of Compound 9(a, b)

(9-1-1) Preparation of Compound 9(a) (24S, Z=t-BuMe₂SiO)

N-methyl morpholine N-oxide (200 g, 1.48 mol), selenium dioxide (38.8 g, 0.35 mol), imidazole (178 g, 2.62 mol), and acetonitrile (4.5 L) are dissolved in dichloromethane (9 L). The compound. 8(a) (24S, Z=t-BuMe₂SiO, d.e.=99%) (446 g, 0.87 mol) prepared in example 8-1 is added to the mixed dichloromethane solution. Then the mixed dichloromethane solution is heated to reflux for 120 minutes. After the reaction is complete base on TLC analysis(with an eluent of 30% ethyl acetate in hexane), the reacted solution is cooled. Then the cooled reacted solution is added with dichloromethane and water for extraction. The organic layer is washed with water, concentrated to give a crude product, Which is purified through a chromatographic column (with an eluent of 6% ethyl acetate in hexane) to give compound 9(a) (24S, Z=t-BuMe₂SiO) (287 g) is obtained.

Compound 9(a)(24R, Z=t-BuMe₂SiO): λₘₐₓ=266 nm. ¹H NMR (200 MHz, CDCl₃) δ 3.43(br, 1H), 4.17 (br, 1H,), 4.49(br,1H), 4.92 (s, 1H), 5.05 (s, 1H), 5.43˜5.53(m, 2H ), 5.83 (d, 1H, J =11.2Hz ), 6.48 (d, 1H, J=11.2 Hz).

(9-1-2) Preparation of Compound 9(a) (24S, Z=t-BuMe₂SiO)

N-methyl morpholine N-oxide (2.0 g, 14.82 mmol), selenium dioxide (0.40 g, 3.60 mmol), triethylamine (0.87 g, 8.60 mmol), and acetonitrile (13.2 mL) are dissolved in dichloromethane (26.4 mL). The compound 8(a) (24S, Z=t-BuMe₂SiO, d.e.=99%) (4.4 g, 8.61 mmol) prepared in example 8-1 is added to the mixed dichloromethane solution. Then the mixed dichloromethane solution is heated to reflux for 180 minutes. The subsequent steps for reaction and purification are the same with that processed in example 9-1-1. After purification by column chromatography compound 9(a) (24S, Z=t-BuMe₂SiO) (1.80 g) is obtained.

(9-1-3) Preparation of Compound 9(a) (24S, Z=t-BuMe₂SiO)

N-methyl morpholine N-oxide (2.0 g, 14.81 mmol), selenium dioxide (0.40 g, 3.60 mmol), and acetonitrile (13.2 mL) are dissolved in dichloromethane (26.4 mL). The compound 8(a) (24S, Z=t-BuMe₂SiO, d.e.=99%) (4.4 g, 8.61 mmol) prepared in example 8-1 is added to the mixed dichloromethane solution. Then the mixed dichloromethane solution is heated to reflux for 180 minutes. The subsequent steps for reaction and purification are the same with that processed in example 9-1-1. After purification by column chromatograpy, compound 9(a) (24S, ZZ=t-BuMe₂SiO) (1.35 g) is obtained

(9-1-4) Preparation of Compound 9(a) (24S, Z=t-BuMe₂SiO)

The compound 8(a) (24S, Z=t-BuMe₂SiO, d.e.=99%) (1.0 g, 1.96 mmol) prepared in example 8-1 is dissolved in methanol (30 mL) under nitrogen or argon atmosphere. Solids of sodium metaperiodate (800 mg) and selenium dioxide (280 mg, 2.52 mmol) are added to the methanol solution. The mixed methanol solution is stirred and heated to reflux for 180 minutes. After the reaction is completed, the reacted solution is cooled and concentrated. Then the cooled reacted solution is added with dichloromethane and water for extraction. The organic layer is washed, and concentrated to give a crude product, which is purified through a chromatographic column (with an eluent of 6% ethyl acetate in hexane) to give compound 9(a) (24S, Z=t-BuMe₂SiO) (0.5 g).

(9-1-5) Preparation of Compound 9(a) (24S, Z=t-BuMe₂SiO)

The compound 8(a) (24S, Z=t-BuMe₂SiO, d.e.=99%) (1.0 g, 1.96 mmol) prepared in example 8-1 is dissolved in ether (25 mL). t-Butyl hydroperoxide (345 μL) and cyclic selenite(cyclic selenite of 1,2-dihydroxy -3-methyl-butane) (121 μl) are added to the ether solution under nitrogen or argon atmosphere to react for 4 hours. The subsequent steps for reaction and purification are the same with that processed in example 9-1-1. After the purification by column chromatography i compound 9(a) (24S, Z=t-BuMe₂SiO) (0.3 g) is obtained.

(9-1-6) Preparation of Compound 9(a) (24S, Z=t-BuMe₂SiO)

The compound 8(a) (24S, Z=t-BuMe₂SiO, d.e.=99%) (0.1 g, 0.2 mmol) prepared in example 8-1 is dissolved in ether (2.5 mL). t-Butyl hydroperoxide (34.5 μL) and cyclic selenite (cyclic selenite of ethylene glycol) (121μL) are added to the ether solution under nitrogen or argon atmosphere to react for 3 hours. The subsequent steps for reaction and purification are the same with that processed in example 9-1-1. After the purification by column chromatography, compound 9(a) (24S, Z=t-BuMe₂SiO) (50 mg) is obtained.

(9-1-7) Preparation of Compound 9(a) (24R, Z=t-BuMe₂SiO)

The compound 8(a) (24R, Z=t-BuMe₂SiO, d.e.=88%) (0.1 g, 0.2 mmol) prepared in example 8-1 is dissolved in a mixed solution of methanol and hexane (methanol:hexane=3:1). The mixed solution is heated to reflux. Diethyl selenite and sodium metaperiodate (500 mg) are added to the refluxed solution. After the reaction is completed, the reacted solution is cooled and concentrated. Then the cooled reacted solution is added with dichlorometlane and water for extraction. The organic layer is washed, and concentrated to give a crude product. After the raw product which is purified through a chromatographic column (with an eluent of 6% ethyl acetate in hexane), a compound 9(a) (24R, Z=t-BuMe₂SiO) (20 mg) is thus obtained

(9-1-8) Preparation of Compound 9(a) (24S, Z=t-BuMe₂SiO)

The compound 8(a) (24R, Z=t-BuMe₂SiO, d.e.=99%) (0.1 g, 0.2 mmol) prepared in example 8-1 is dissolved in a mixed solution of methanol and hexane (methanol:hexane=3:1). Diethyl selenite and 70% ter-butyl hydroperoxide (400 mg) aqueous solution are added to the mixed solution. The mixed solution is stirred at room temperature until the reaction is completed. The subsequent steps for reaction and purification are the same with that processed in example 9-1-7. After purification by column chromatography compound 9(a) (24S, Z=t-BuMe₂SiO) (30 mg) is obtained.

(9-1-9) Preparation of Compound 9(a) (24S, Z=t-BuMe₂SiO)

The compound 8(a) (24R, Z=t-BuMe₂SiO, d.e.=99%) (0.1 g, 0.2 mmol) prepared in example 8-1, diethyl selenite (250 μL ), and N-methyl morpholine N-oxide (500 mg) are dissolved in a mixed solution of methanol and hexane (methanol:hexane=3:1). The subsequent steps for reaction and purification are the same with that processed in example 9-1-7. After purification by column chromatography, a compound 9(a) (24S, Z=t-BuMe₂SiO) (20 mg) is obtained.

(9-1-10) Preparation of Compound 9(a) (24S, Z=t-BuMe₂SiO)

The compound 8(a) (24R, Z=t-BuMe₂SiO, d.e.=99%) (2.5 g, 4.89 mmol) prepared in example 8-1, N-methylmorpholine N-oxide (1.25 g, 9.26 mmol), and water (1.7 mL) are dissolved in THF (50 mL). Selenious acid (1.25 g) and an acetonitrile solution (1.25 mL) of N-methyl-morpholine (1.25 g) are added to the mixed THF solution. The subsequent steps for reaction and purification are the same with that processed in example 9-1-7. After purification by column chromatography compound 9(a) (24S, Z=t-BuMe₂SiO) (0.5 g) is obtained.

(9-2) Preparation of Compound 9(b) (24R, Z=t-BuMe₂SiO)

The steps for the preparation are similar to those described in example (8-1) except that the reactant, i.e. compound 8(a) (24S, Z=t-BuMe₂SiO, d.e.=99%), for the reaction is replaced by compound 8(b)(24R, Z=t-BuMe₂SiO). The amounts of the related reagent are also adjusted. After the purification is achieved, compound 9(b) (24R, Z=t-BuMe₂SiO) (48 mg) is obtained.

### EXAMPLE 10

### Preparation of Compound 1(a, b)

(10-1-1) Preparation of Compound 1(a)(1α,3β,5Z,7E,22E,24S) and 1(a),(1β,3β,5Z,7E,22E,24S)

Compound 9(a) (200 g, 0.38 mol) (24S,Z=t-BuMe₂SiO) is tetra-n-butylammonium fluoride (0.36 kg, 1.14 mol ) are dissolved in THF (2.7 L). Then the mixed solution is heated to reflux. After the reaction is complete, the reaction solution is concentrated. The residue after concentration is added with ethyl acetate and water for extraction. The organic layer is washed, concentrated to give a crude product, which is purified through a chromatographic column to give compound 11(a) [1(α,β),3β,5E,7E,22E,24S] (130 g) is obtained.

The compound 11(a) [1(α,β),3β,5E,7E,22E,24S](130 g) is added to a mixture of ethyl acetate (130 mL) and water (520 mL). The mixture is stirred for 1 hour at room temperature. Then the mixture is filtered and a solid product 11(a) [1(α,β),3β,5E,7E,22E,24S] (80 g) is obtained.

The filtrate is separated and the organic layer is separated and concentrated to obtain a residue (50 g).

The residue, and phenyl boronic acid (10 g, 82 mmol) are dissolved in dichloromethane (2 L). The reaction is processed for 3.5 hours. After the reaction is complete, the reacted solution is concentrated and purified through a chromatographic column to give compound 11(a) [1α,3β,5E,7E,22E,24S] (15 g) and cyclic-1,3-boronate ester of 11(a) [1β,3β,5E,7E,22E,24S] (15 g).

The compound 11(a) [1α,3β,5E,7E,22E,24S] (61 g), and 9-acetylanthracene (6 g) are dissolved in acetone (10 L). The acetone solution is photolysis at a temperature less than 10° C. in an atmosphere of argon. After the reaction is complete, the reaction mixture is concentrated and purified through a chromatographic column to give a crude product (compound 1(a) [1α,3β,5E,7E,22E,24S] (60.1 g).

Compound 1(a) [1α,3β,5E,7E,22E,24S]: λₘₐₓ=266 nm. m/z: 412. ¹H NMR (200 MHz, CDCl₃) δ 0.54 (s, 3 H), 1.02 (d, 3H, J=8.25 Hz), 3.39-3.43(m, 1H), 4.20(br, 1H), 4.41(br, 1 H), 4.97(s, 1 H), 5.29(s, 1 H), 5.43-5.46 (m, 2 H),5.99 (d, 1H, J=14.0 Hz ),6.35 (d, 1 H, J=14.05 Hz).

The cyclic-1,3-boronate ester of 11(a) [1β,3β,5E,7E,22E,24S] (10 g) produced is dissolved in ethyl acetate. Then hydrogen peroxide (10 ml) is added to the ethyl acetate solution to react for 1 hour. After the reaction is completed, the reaction mixture is concentrated and purified through a chromatographic column to give compound 11(a) [1β,3β,5E,7E,22E,24S] (5 g).

The compound 11(a) [1β,3β,5E,7E,22E,24S] (5 g, 12.12 mmol) produced, and 9-acetylanthracene (0.5 g, 2.27 mmol) are dissolved in acetone in an atmosphere of argon. The acetone solution is photolysis at a temperature less than 10° C. After the reaction is completed, the reacted solution is concentrated and purified through a chromatographic column to give compound 1(a) [1β,3β,5E,7E,22E,24S].

Compound 1(a) [1β,3β,5E,7E,22E,24S]: λₘₐₓ=266 nm. m/z: 412. ¹H NMR (200 MHz, CDCl₃) δ 0.54 (s, 3 H ), 1.0 (d, 3H, J=6.62 Hz), 3.36-3.43 (m, 1H), 4.07(m, 1H), 4.32 (br, 1H), 4.97(s,1H), 5.25(s, 1 H), 5.42-5.45 (m, 2 H), 6.02 (d, 1 H, J=11.24 Hz), 6.41(d, 1 H, J =11.24 Hz).

(10-1-2) Preparation of Compound 1(a)(1α,3β,5Z,7E,22E,24S)

Compound 9(a) (287 g, 0.54 mol) (24S,Z=t-BuMe₂SiO), tetra-n-butylammonium fluoride (344 g, 1.09 mol) are dissolved in THF (2.8 L). Then ,the mixture is heated to reflux. After the reaction is complete,1 the reaction mixture is concentrated. The residue after concentrated is added with ethyl acetate and water for extraction. The organic layer is washed, concentrated to give a crude product, which is purified through a chromatographic column to give compound 11(a) [1(α,β)3β,5E,7E,22E,24S] (139 g).

The compound 11(a) [1(α,β)3β,5E,7E,22E,24S] (139 g, 0.34 mol), and 9-acetylanthracene (13.9 g, 63.10 mmol) are dissolved in acetone (20 L) in an atmosphere of argon. The mixture is photolysis at a temperature less than 10° C. After the reaction is complete, the reaction mixture is concentrated and purified through a chromatographic column to give compound 1(a) [1(α,β)3β,5E,7E,22E,24S] (60.1 g).

The compound 1(a) [1(α,β)3β,5E,7E,22E,24S] and phenyl boronic acid (16 g, 0.13 mmol) are dissolved in acetone (5 L). The reaction is processed for 3 hours. After the reaction is complete, the reaction mixture is concentrated and purified through a chromatographic column to give compound 1(a) [1α,3β,5E,7E,22E,24S] (126 g) and cyclic-1,3-boronate ester of 1(a) [1β,3β,5E,7E,22E,24S].

(10-1-3) Preparation of Compound 1(a)(1α,3β,5Z,7E,22E,24S)

The steps of the preparation are similar to those processed in example (10-1-1). However, the sequence of the steps is changed. In the present example, the photolysis reaction for isomerization is proceeded first, then the de-protection on C3, reaction with phenyl boronic acid, and purification for separating C1(α,β) is proceeded subsequently.

A product compound 1(a) can be obtained after the staring material compound 9(a) (24S, Z=t-BuMe₂SiO)(10 g, 19 mmol) prepared in example (9-1-1) is proceeded through the reaction illustrated above.

(10-2) Preparation of Compound 1(b)(1α,3β,5Z,7E,22E,24R) and 1(b)(1β,3β,5Z,7E,22E,24R)

The steps for the preparation of compound 1(b)(1α,3β,5Z,7E,24R) and 1(b)(1β,3β,5Z,7E,24R) are similar to those described in example (10-1-1) except that the reactant, i.e. compound 1(a), for the reaction is replaced by compound 9(b) prepared in example (9-2). The steps of other reaction are similar to those processed in example (10-1-1). The sequence of the photolysis reaction for isomerization is proceed, the de-protection on C3, and the purification for separating C1(α,β) are similar to the example (10-1-1).

### EXAMPLE 11

### Crystallization of Compound 1(a) (1α,3β,5Z,7E,22E,24S)

Compound 1(a) (10 g, 0.38 mol) (1α,3β,5Z,7E,22E,24S) is dissolved in methanol (40 mL). After the methanol solution is filtered and concentrated, another 40 mL of acetone is added. The solution is stirred at room temperature for 1 hour and washed by 10 mL of acetone. After washing is achieved, solid product is obtained. The solid product is vacuumed dried at a temperature of 30° C. Then crystals of compound 1(a) (1α,3β,5Z,7E,22E,24S) is obtained. Although the present invention has been explained in relation to its preferred embodiment, it is to be understood that many other possible modifications and variations can be made without departing from the spirit and scope of the invention as hereinafter claimed.

1. A method for preparing a C1,C24-dihydroxyl-vitamin D, comprising the following steps:
(a) oxidizing a starting material of formula (1): 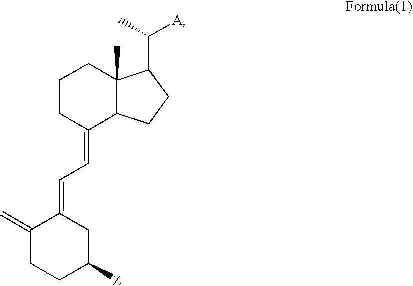
by an oxidant in the present of a base to form a mixture of isomers, wherein A is 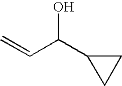
or 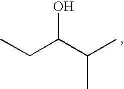
and Z is a protected hydroxyl group; and
(b) photo-isomerizing and deprotecting the mixture of isomers to form a C1(α)-hydroxyl-C24-hydroxyl-vitamin D or C1(β)-hydroxyl-C24-hydroxyl-vitamin D of formula (2): 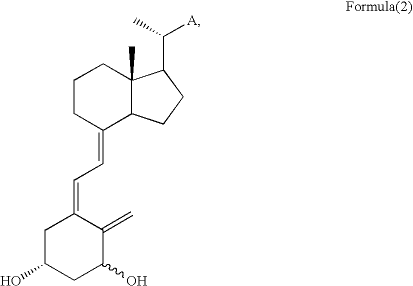
wherein the oxidant is a selenium dioxide or a selenite ester of formula (3):
R⁶O—Se(O)—OR⁷ (3)
wherein R⁶ and R⁷ are individually hydrogen, C₁-C₉ alky, C₁-C₉ aralkyl, or the combination thereof, and R⁶ and R⁷ are identical or different.

2. The method as claimed in claim 1, wherein R⁶ and R⁷ are individually hydrogen, C₁-C₄ alkyl.

3. The method as claimed in claim 1, wherein the starting material is oxidized in the presence of a co-oxidant in step (a).

4. The method as claimed in claim 3, wherein the starting material is oxidized in an organic solution comprising the oxidant, the co-oxidant, and the base dissolved in an organic solvent.

5. The method as claimed in claim 3, wherein the co-oxidant is a metal salt of a peracid, an alkyl hydroperoxide in which the alkyl moiety contains from 4 to 16 carbon atom, a non-aromatic tertiary amine oxide, or a combination thereof.

6. The method as claimed in claim 5, wherein the co-oxidant is sodium metaperiodate.

7. The method as claimed in claim 5, wherein the co-oxidant is N-methylmorpholin N-oxide.

8. The method as claimed in claim 4, wherein the organic solvent is alkanol having 1 to 9 carbon atoms, haloalkane having 1 to 9 carbon atoms, alkyl nitril having 1 to 9 carbon atoms, aromatic hydrocarbons having 6 to 9 carbon atoms, or the combination thereof.

9. The method as claimed in claim 4, wherein the oxidant is selenium dioxide, and the co-oxidant is N-methylmorpholin N-oxide.

10. The method as claimed in claim 1, wherein the photo-isomerization is a photoreaction initiated by a photosensitizer.

11. The method as claimed in claim 10, wherein the photosensitizer is anthracene or a derivative thereof, phenazine or a derivative thereof, acridine or aderivative thereof, or the combination thereof.

12. The method as claimed in claim 1, wherein the deprotection is processed by a deprotecting reagent of quaternary amine salt.

13. The method as claimed in claim 12, wherein the quaternary amine salt is tetra-n-butylammonium fluoride.

14. The method as claimed in claim 1, wherein “Z” is an ether or ester.

15. The method as claimed in claim 1, wherein the ether is tert-butyldimethylsilyloxy.

16. The method as claimed in claim 1, wherein the starting material is [5E,7E,22E,24R]-24-cyclopropyl-3β-(tert-butyldimethyl-silyloxy)-9,10-secochola-5,7,10(19),22-tetraene-24-ol, [5E,7E,22E,24S]-24-cyclopropyl-3β-(tert-butyldimethylsilyloxy)-9,10-secochola -5,7,10(19),22-tetraene-24-ol, or the combination thereof.

17. The method as claimed in claim 1, wherein the starting material is [5E,7E,24R]-24-isopropyl-3β-(tert-butyldimethylsilyloxy)9,10-secochola-5,7,10(19)-triene-24-ol, [5E,7E,24S]-24-isopropyl-3β-(tert-butyldimethylsilyloxy)-9,10-secochola-5,7,10(19)-triene-24-ol, or the combination thereof.

18. The method as claimed in claim 1, wherein the isomer is [5E,7E,22E,24R,]-24-cyclopropyl-3β-(tert-butyidimethylsilyloxy)-9,10-secochola-5,7,10(19),22-tetraene-1(α,β),24-diol, [5E,7E,22E,24S,]-24-cyclopropyl-3β-(tert-butyldimethylsilyloxy)-9,10-secochola-5,7,10(19),22-tetraene-1(α,β),24-diol, or the combination thereof.

19. The method as claimed in claim 1, wherein the isomer is [5E,7E,24R]-24-isopropyl-3β-(tert-butyldimethylsilyloxy)-9,10-secochola-5,7,10(19)-triene -1(α,β),24-diol, [5E,7E,24S]-24-isopropyl-3β-(tert-butyldimethylsilyloxy)-9,10-secochla-5,7,10(19)-triene-1(α,β),24-diol, or the combination thereof.

20. The method as claimed in claim 1, wherein the C1(α,β),C24-trihydroxyl-vitamine D is (5Z,7E,22E,24R)-24-cyclopropyl-9,10-secochola-5,7,10(19),22-tetraene-1(α,β),3β,24-triol, (5Z,7E,22E,24S)-24-cyclopropyl-9,10-secochola-5,7,10(19),22-tetraene-1(α,β),3β,24-triol, and the combination thereof.

21. The method as claimed in claim 1, wherein the C1(α,β),24-trihydroxyl-vitamine D is (5Z,7E,24R)-24-isopropyl-9,10-secochola-5,7,10(19)-triene-1(α,β),3β,24-triol or (5Z,7E,24S)-24-isopropyl-9,10-secochola-5,7,10(19)-triene-1(α,β),3β,24-triol.

22. A method for preparing a C1α,C3,C24-trihydroxyl-vitamin D and a C1β,C3,C24-trihydroxyl-vitamin, comprising the following steps:
(a) oxidizing a starting material of formula (1): 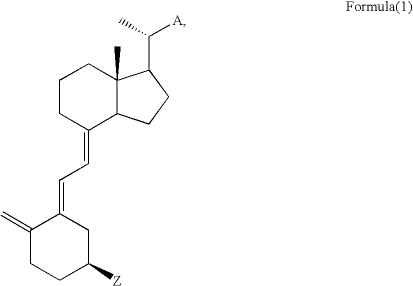
by an oxidant in the presence of a base to form a mixture of isomers, wherein A is 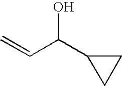
or 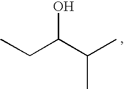
and Z is a protected hydroxyl group;
(b) photo-isomerizing and deprotecting the mixture of isomers, and forming a mixture of complexes with a ligand, wherein a mixture of the complexes is formed after deprotection, and the ligand is alkyl boronic acid or aryl boronic acid; and
(c) separating the C1α,C3,C24-trihydroxyl-vitamin D, and the C1β, C3,C24-trihydroxyl-vitamin D complexed with the ligand from the mixture of the complexes;
wherein the oxidant is a selenium dioxide or a selenite ester of formula (3)
Rt⁶O—Se(O)—OR⁷ (3)
wherein R⁶ and R⁷are individually hydrogen, C₁-C₉ alkyl, C₁-C₉ aralkyl, or the combination thereof, and R⁶ and R⁷ are identical or different.

23. The method as claimed in claim 22, wherein the aryl boronic acid is phenylboronic acid.

24. The method as claimed in claim 22, wherein the photo-isomerization is proceeded before the deprotection and formation of complexes, or after the deprotection and the formation of the complexes.

25. The method as claimed in claim 22, wherein the photo-isomerization is processed between the deprotection and formation of complexes.

26. The method as claimed in claim 22, wherein the starting material is oxidized in the presence of a co-oxidant.

27. The method as claimed in claim 26, wherein the starting material is oxidized in an organic solution comprising the oxidant, the co-oxidant, and the base dissolving in an organic solvent.

28. The method as claimed in claim 22, wherein the C1β,C3,C24-trihydroxyl-vitamin D complexed with the boronic acid ligand is (5Z,7E,22E,24R)-24-cyclopropyl-9,10-secochola-5,7,10(19),22-tetraene-1β,3β,24-triol, (5Z,7E,22E,24S)-24-cyclopropyl-9,10-secochola-5,7,10(19),22-tetraene-,1β,3β,24-triol, or the combination thereof.

29. The method as claimed in claim 26, wherein the C1β,C3,C24-trihydroxyl-vitamin D complexed with the boronic acid ligand is (5Z,7E,24R)-24-isopropyl-9,10-secochola-5,7,10(19)-triene-1β,3β,24-triol, (5Z,7E,24S)-24-isopropyl-9,10-secochola-5,7,10(19)-triene-1β,3β,24-triol or the combination thereof.

30. The method as claimed in claim 22, wherein the C1β,C3,C24-trihydroxyl-vitamin D complexed with the boronic acid ligand is (5E,7E,22E,24R-24-cyclopropyl-9,10-secochola-5,7,10(19),22-tetraene-1β,3β,24-triol, (5E,7E,22E,24S)24-cyclopropyl-9,10-secochola-5,7,10(19),22-tetraene-1β,3β,24-triol, or the combination thereof.

31. The method as claimed in claim 22, wherein the C1β,C3,C24-trihydi-oxyl-vitamin D complexed with the boronic acid ligand is [5E,7E,24R]-isopropyl-9,10-secochola-5,7,10(19)-triene-1β,3β,24-triol, [5E,7E,24R]-24-isopropyl-9,10-secochola-5,7,10(19)-triene-1β,3β,24-triol, or the combination thereof.

32. A method for separating a C1(α,β),C3,C24-trihydroxyl-vitamin D, comprising following steps:
(a) providing a mixture of isomers of the 1,3,C24-trihydroxyl-vitamin D;
(b) reacting the mixture of the isomers of the 1,3,C24-trihydroxyl-vitamin D with a ligand to form a ring-structured complex, wherein the lignad is alkyl boronic acid or aryl boronic acid, and the 1,3,C24-trihydroxyl-vitamin D to form a ring-structured complex is an 1,3-cisC24-trihydroxyl-vitamin D; and
(c) separating the ring-structure-complexed analog of 1,3-cis-C24-trihydroxyl-vitamin D from the 1,3-trans-C24-trihydroxyl-vitamin D, and reduce the ring-structure-complexed 1,3-cis-C24-trihydroxyl-vitamin D from the 1,3-trans-C24-trihydroxyl-vitamin D to obtain the C1β,C3β,C24-trihydroxyl-vitamin D and the C1α,C3,C24-trihydroxyl-vitamin D, individually;
wherein the isomer of the 1,3,C24-trihydroxyl-vitamin D is a compound having a structure of formula (2) 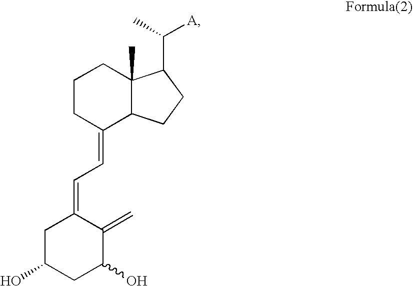
or having a structure of formula (4) 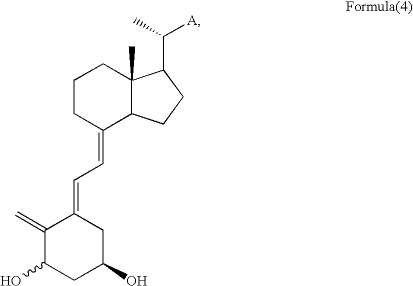
wherein A is

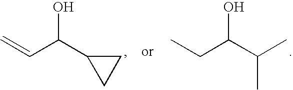

33. The method as claimed in claim 32, wherein the aryl boronic acid is phenylboronic acid.
